(19) United States
(12) Patent Application Publication
(10) Pub. No.: US 2025/0114458 A1
(43) Pub. Date: Apr. 10, 2025

(54) NIR BIOSAFE QR CODES WITH BIOINKJET PRINTING AND TAGGING

(71) Applicant: Purdue Research Foundation, West Lafayette, IN (US)

(72) Inventors: Young Kim, West Lafayette, IN (US); Joshua Kim, West Lafayette, IN (US); Jungwoo Leem, West Lafayette, IN (US)

(73) Assignee: Purdue Research Foundation, West Lafayette, IN (US)

(21) Appl. No.: 18/885,648

(22) Filed: Sep. 14, 2024

Related U.S. Application Data
(60) Provisional application No. 63/543,026, filed on Oct. 6, 2023.

(51) Int. Cl. A61K 47/16; A61K 9/00; A61K 9/20; A61K 47/08; A61K 47/20

(52) U.S. Cl. CPC A61K 47/16; A61K 9/0053; A61K 9/2013; A61K 47/08; A61K 47/20

(57) ABSTRACT

A method of providing an edible printer dye for use as a predetermined pattern including a stealth quick response (QR) code taggant for an ingestible food or pharmaceutical product includes dissolving one or more edible infrared (IR) or near IR (NIR) light absorbing dyes in a carrier solution based on a predetermined concentration of the one or more IR or NIR light absorbing dyes, thereby generating an IR or NIR light absorbing dye solution, adding a mixture of dispersing agents to the dye solution, thereby generating an edible IR or NIR light absorbing dyes, loading the edible IR or NIR light absorbing dyes in any one of a printer cannisters, and loading other visible edible dyes in corresponding other printer cannisters.

### CROSS-REFERENCE TO RELATED APPLICATIONS

The present non-provisional patent application is related to and claims the priority benefit of U.S. Provisional Patent Application Ser. 63/543,026, filed Oct. 6, 2023, the contents of which are hereby incorporated by reference in its entirety into the present disclosure.

### STATEMENT REGARDING GOVERNMENT FUNDING

None.

### TECHNICAL FIELD

The present disclosure generally relates to a system and method of tagging ingestible products, and in particular to a system and method for near-infrared (NIR) light absorbing tag placement on the ingestible product.

### BACKGROUND

This section introduces aspects that may help facilitate a better understanding of the disclosure. Accordingly, these statements are to be read in this light and are not to be understood as admissions about what is or is not prior art.

Fake pharmaceutical drugs pose a ubiquitous challenge including a considerable threat to public and patient safety worldwide. Fake and counterfeit medicines include a wide array of substandard, falsified, and diverted pharmaceutical products. The World Health Organization (WHO) estimates that over one million people die annually because of counterfeit pharmaceutical products and that several disasters can be attributed to poor medicine security. For example, counterfeit malaria and pneumonia medications have been estimated to result in 250,000 child deaths annually in Africa. Cambodia witnessed the loss of 30 lives because of counterfeit antimalarial drugs that included outdated components. In Nigeria, over 50,000 individuals were administered fraudulent meningitis vaccines, leading to a death toll of 2,500. The consumption of cough syrups laced with paracetamol-containing diethylene glycol resulted in 89 deaths in Haiti, and similar cough syrups were linked to the tragic demise of 30 infants in India. Recently, the sale of counterfeit pharmaceuticals containing illicit substances in Mexican pharmacies has made its way into the United States.

The proliferation of online (or internet) pharmacies has intensified the illicit trade of medications across the global, national, and local levels. Globally, approximately 40,000 online pharmacies exist; however, a strikingly low 3% of them operate legally. Evidently, there has been a surge in counterfeit medical treatments and supplies amid the COVID-19 pandemic driven by heightened demand. Social media and online platforms allow even high school students to acquire controlled substances (e.g., OxyContin, Vicodin, Xanax, and Adderall) along with counterfeit renditions. An estimated 18% of high school students have access to controlled substances without a prescription. Drug dealers target teenagers by distributing counterfeit medicines in the form of legitimate prescriptions. Recently, an increase in accidental youth deaths has been closely associated with the proliferation of illicit fentanyl distributed on Snapchat and Instagram. Unfortunately, there is lack of public awareness regarding the perils associated with counterfeit medicines, and therefore, it is imperative to implement advanced medicine security technologies.

Pharmaceutical companies conventionally apply anticounterfeit measures that rely on the exterior box (e.g., secondary packaging) used to pack multiple medicine products together. However, exterior/secondary box-level protection is fundamentally limited because medicines must be removed from the initial packaging and separated into individual doses for sale and at—home use, often rendering exterior box-level protection useless. As a result, each medicine cannot be individually verified or authenticated when separated from the secondary package. Unique objects, barcodes, quick response (QR) codes, radiofrequency identification, and holograms are commonly used for product integrity and brand protection with a focus on supply chains. In the United States, the Drug Supply Chain Security Act has mandated the establishment of unit-level traceability by 2023. Therefore, many US pharmaceutical manufacturers, retailers, and distributors have agreed to create blockchain technology for better managing complex pharmaceutical supply chains (also known as the MediLedger Network).

Several dose-level authentication methods for medicines and pharmaceutical products have been developed (see Table 1 for comprehensive comparisons); for example, silica microtaggants, DNA taggants, plasmonic nanotags, multicolor nonpareil coatings, all-protein-based physical unclonable functions, watermark bioprinting, QR code drug labels, and matrix codes. However, the existing dosage-level identification methods have several limitations: First, the constituent materials are not easily accessible, procured, or fabricated on a large scale. Second, some materials used in on-dose technologies may not be safe for oral intake because of the potential hazards and cytotoxicity associated with artificial and foreign materials. Further, there are concerns regarding certain medicine-printing materials (e.g., phthalates) that possess endocrine-disrupting properties. Third, the existing on-dose technologies often require skilled and trained personnel, as well as high-cost sophisticated analyzers and specialized readers equipped with optical components. Meanwhile, the QR code-based authentication offers not only simple and effective data storage for speedy retrieval but is also robust against variations in illumination, scale, coverage, and camera angles for reading. This method enables end users (e.g., patients and healthcare professionals) to extract data via online or offline modes on Android and iOS smartphones. However, a major drawback of this approach is the reduced security: QR codes can be duplicated and copied easily because of their visibility.

TABLE 1
Comparisons of on-dose authentication technologies for medicines

| | | | | | | Fabrication |
|---|---|---|---|---|---|---|
| | | | | | | and |
| | | | | | | reading |
| | Fabrication | Main | | Reading | | apparatus |
| Type | method | material | Toxicity | apparatus | Clonability | cost |
| Microtaggants | Microparticles | Silica | Low, but | Mobile lab- | Difficult | High |
| | (Chemical | | reported | based reader | | |
| | synthesis) | | | and/or | | |
| | | | | smartphone | | |
| | | | | camera | | |
| DNA | DNA | Synthetic | Low | DNA qPCR | Difficult | High |
| molecular | molecular | DNA | | instrument | | |
| tags | cloning | | | | | |
| Plasmonic | Nanoparticles | Gold | Low, but | Raman | Difficult | High |
| nanotags | (Colloidal | (Au) | reported | spectrometer | | |
| | synthesis) | | | | | |
| Nonpareils | Particles | Nonpareils | Low | Smartphone | Medium | Low |
| color | (Synthesis) | (Food | | camera | | |
| spheres | | colorings, | | (Customized | | |
| | | sugar, and | | app) | | |
| | | starch) | | | | |
| Physical | Microparticles | Fluorescent | Low | Smartphone | Difficult | Medium |
| unclonable | and films | silk | | camera | | |
| function | (Dissolving, | fibroin | | equipped | | |
| | freeze | | | light sources | | |
| | drying, and | | | and optical | | |
| | casting) | | | filters | | |
| 2D matrix | Thin films | Fluorescent | Low | Smartphone | Difficult | Medium |
| code | (Dissolving | silk | | camera | | |
| | and | fibroin | | equipped | | |
| | casting) | | | light sources | | |
| | | | | and optical | | |
| | | | | filters | | |
| Watermarking | Inkjet | Food | Low | Smartphone | Difficult | Medium |
| | printing | colorings, | | camera | | |
| | | fluorescent | | (Customized | | |
| | | silk | | app) | | |
| | | fibroin | | | | |
| QR code | Soft | Poly(ethylene | Low, but | Smartphone | Easy | High |
| | lithography | glycol) | reported | camera | | |
| | (microfluidics) | diacrylate | | (Built-in QR | | |
| | and | (PEG-DA), | | code reader | | |
| | digital | rhodamine | | app) | | |
| | mirror | B | | | | |
| | device | | | | | |
| | (DMD) | | | | | |
| QR code | Soft | Hyaluronic | Low | Smartphone | Easy | High |
| | lithography | acid | | camera | | |
| | | (HA), | | (Built-in QR | | |
| | | gelatin, | | code reader | | |
| | | polyvinyl | | app) | | |
| | | alcohol | | | | |
| | | (PVA) | | | | |
| QR code | Chemical | Upconversion | High | Smartphone | Difficult | High |
| | synthesis | fluorescent | | camera | | |
| | and | nanoparticles | | (Customized | | |
| | inkjet | | | app) | | |
| | printing | | | | | |
| QR code | Inkjet | Food | Low | Smartphone | Easy | Low |
| | printing | coloring, | | camera | | |
| | | hydroxypropyl | | (Built-in QR | | |
| | | methylcellulose | | code reader | | |
| | | (HPMC) | | app) | | |

Therefore, there is an unmet need for a novel robust method and system allowing the placement of a tag on ingestible products that can be used to verify the authenticity of the product.

### SUMMARY

A method of providing an edible printer dye for use as a predetermined pattern including a stealth quick response (QR) code taggant for an ingestible food or pharmaceutical product is disclosed. The method includes dissolving one or more edible infrared (IR) or near IR (NIR) light absorbing dyes in a carrier solution based on a predetermined concentration of the one or more IR or NIR light absorbing dyes, thereby generating an IR or NIR light absorbing dye solution. The method further includes adding a mixture of dispersing agents to the dye solution, thereby generating an edible IR or NIR light absorbing dyes. Additionally, the method includes loading the edible IR or NIR light absorbing dyes in any one of a printer cannisters, and loading other visible edible dyes in corresponding other printer cannisters.

Another method of generating an edible and stealth predetermined pattern on an ingestible product is disclosed. The method includes printing a predetermined pattern including a quick response (QR) code onto an edible substrate, and adhering the substrate to an ingestible product, the predetermined pattern having a pattern derived from: one or more visible light absorbing dyes, and one or more edible infrared (IR) or near IR (NIR) light absorbing dyes, wherein the one or more edible IR or NIR light absorbing dyes are based on dissolving one or more edible IR or NIR light absorbing dyes in a carrier solution based on a predetermined concentration of the one or more edible IR or NIR light absorbing dyes, thereby generating an edible IR or NIR light absorbing dye solution, and adding a mixture of dispersing agents to the dye solution, thereby generating one or more edible IR or NIR light absorbing dyes.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1a-1c, are schematics showing generation of the QR code taggant (FIG. 1a), application of the taggant to an ingestible product (FIG. 1b), and reading the taggant by a smartphone (FIG. 1c), illustrate the concept of a camouflaged QR code taggant using edible and biologically safe inks and substrates via inkjet printing for protecting individual ingestible products at an individual product level, according to the present disclosure.

FIG. 2a, provides chemical structures of various ink species of interest, according to the present disclosure.

FIGS. 2b and 2c are a photograph (FIG. 2b) and normalized absorption spectra (FIG. 2c) of food colorings and IR820 dye dissolved in deionized water.

FIGS. 2d and 2e, include photographs (FIG. 2d) and normalized absorption spectra (FIG. 2e) of color inks (1:10000 diluted) and IR820 inks.

FIG. 3a includes photographs of QR codes printed on silk fibroin print sheets with different opacity levels of the RGB colors in a range of 20-100%.

FIG. 3b is a graph of success rate in % vs. opacity of printed RGB colors in %.

FIGS. 3c-3d are photographs (FIG. 3c) and scan success rate (FIG. 3d) of QR codes printed on silk fibroin print sheets with different concentrations of IR820 inks in a range of 1-30 mM.

FIG. 4a is a graph of printing reproducibility test of 200 different taggants with the same QR code pattern using bioinkjet printing.

FIG. 4b is a graph showing scan success rates of 12 different QR code taggants printed at different concentrations of IR820 ink for photostability tests.

FIG. 4c is the same as FIG. 4b but for long-term stability tests.

FIG. 4d provides photographs of original and simulated (scan-print copy) QR code taggants.

FIG. 5 is a collection of photographs of a smartphone utilized to simulate authentication process of a QR code taggant affixed to an oral-dosage tablet-type medicine.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 15%, within 10%, within 5%, or within 1% of a stated value or of a stated limit of a range.

In the present disclosure, the term “substantially” can allow for a degree of variability in a value or range, for example, within 85%, within 90%, within 95%, or within 99% of a stated value or of a stated limit of a range.

A novel robust method and system is disclosed herein which allows for placement of a tag on ingestible products that can be used to verify authenticity of the product. Towards this end, a camouflaged biosafe QR code bioprinting and taggant construction is disclosed which can be used as a security measure for ingestible products at an individual ingestible items level. An inkjet printing method was modified, which is scalable for printing edible materials and additive pharmaceutical manufacturing. Further, inkjet printing inks were fabricated using biologically safe and edible materials, such as Food and Drug Administration (FDA)-approved edible coloring and near-infrared (NIR) dyes, for camouflaged effects with increased QR code security. We also constructed edible and digestible natural protein-based print sheets, on which the QR code is printed, and we generated QR codes with color and invisible (hidden) patterns by determining the optimal printing parameters. We evaluated the biocompatibility, enzymatic digestibility, and reliability of the proposed QR codes. In addition, we tested the resistance to copy attack when a scanner or copier is used to duplicate QR codes. Finally, we demonstrated the reading of QR codes affixed to individual ingestible products in a solid dosage format (e.g., tablet, pill, capsule, a fruit, a food item, etc.) using a built-in QR code scanner in a smartphone.

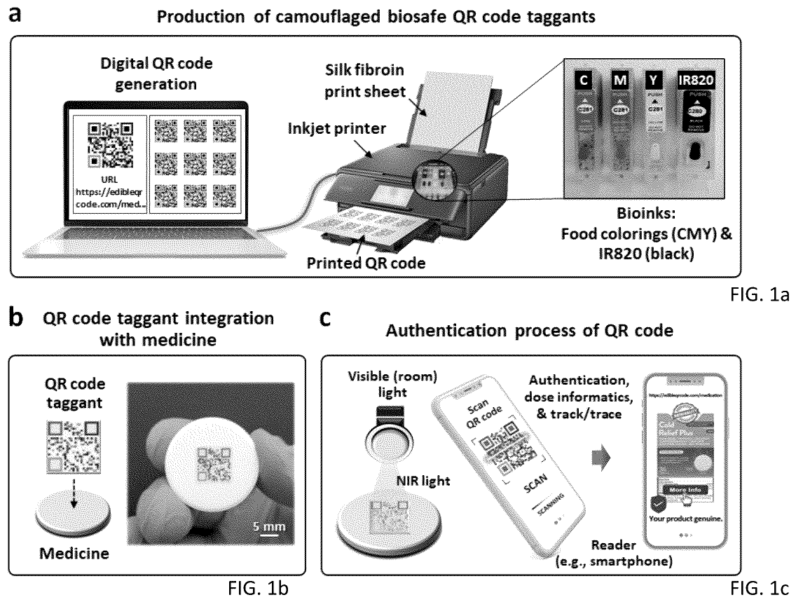

Referring to FIGS. 1a-1c, which are schematics showing generation of the QR code taggant (FIG. 1a), application of the taggant to an ingestible product (FIG. 1b), and reading the taggant by a smartphone (FIG. 1c), illustrate the concept of a camouflaged QR code taggant using edible and biologically safe inks and substrates via inkjet printing for protecting individual ingestible products at an individual product level, according to the present disclosure. The proposed QR code taggant is constructed using bioinkjet printing on a protein print sheet (FIG. 1a), according to one embodiment, or directly onto the ingestible product, according to another embodiment. A QR code is designed using a QR code generator, which allows for the generation of a URL hyperlink or the encoding of product information. The generated digital QR code is printed on a protein (silk fibroin) print sheet using a commercially available inkjet printer with color and invisible NIR inks. FDA-approved food coloring is the main ingredient in the color inks. Indocyanine green (ICG) is used as an NIR-only absorbing ink, according to one embodiment, which is commonly used for medical imaging and cancer treatment because of its low cytotoxicity. Furthermore, ICG combines well with the silk fibroin. We also used IR820 (also known as the new ICG) as an ICG alternative, according to another embodiment of an NIR-only absorbing ink, because it has advantages over conventional ICG, including lower costs and enhanced stability. IR820 is invisible to the naked eye because of its low absorption in the visible wavelength range of 400-650 nm.

The proposed QR code taggant exhibits three features for on-dose authentication. First, the QR code contains an additional invisible hidden pattern as part of the QR code to overcome the security limitations of conventional QR codes. It is unlikely that a counterfeiter will be able to successfully duplicate the entire QR code using a simple scan-print method (i.e., a copy attack). Second, all constituent materials, including silk, are biologically safe and edible. This proposed QR code taggant can be affixed to a solid ingestible product (e.g., tablet or capsule, solid or semi-solid foods such as cheese, cake, chocolate, fruits, and other solid or semi-solid ingestible products) using an edible adhesive by the food or drug manufacturer or pharmacist (FIG. 1b). Third, an end user (e.g., a patient or a consumer) can easily scan the QR code immediately before oral intake using an onboard QR code scanner on a smartphone (FIG. 1c). As part of the authentication, the end user can access additional information on the dose and track/trace, including product data (e.g., dosage strength, dose frequency, cautions, and expiration date), manufacturing details (e.g., location, date, batch, and lot number), and distribution paths (e.g., country, distributor, and wholesaler) in addition to advertisement material including coupons for future purchases.

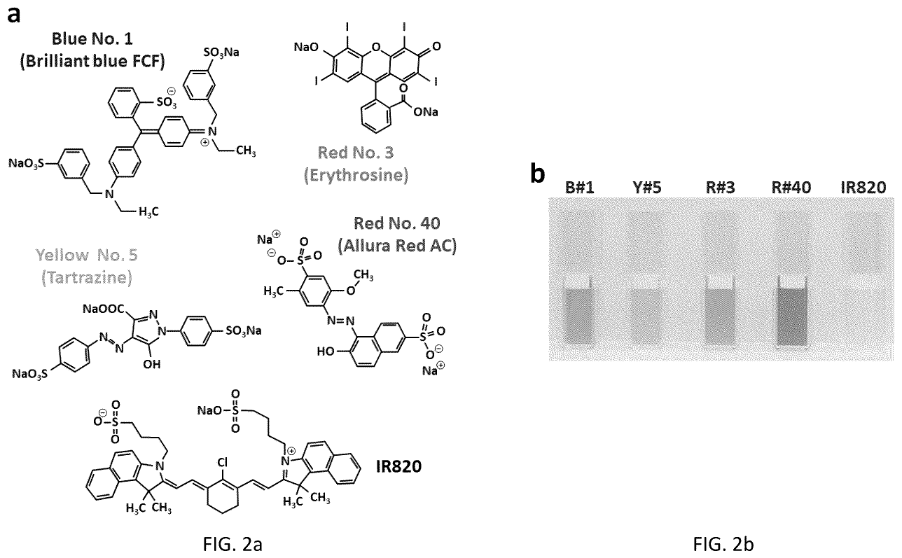

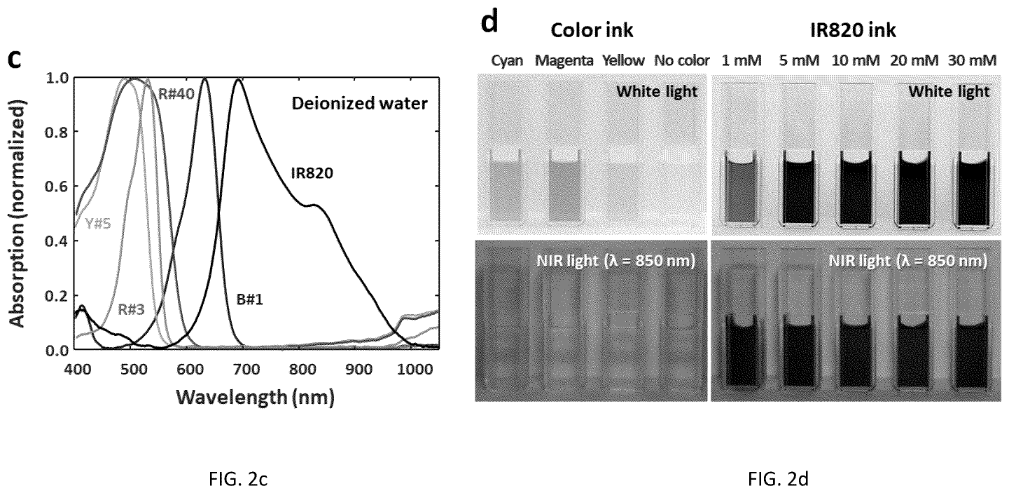

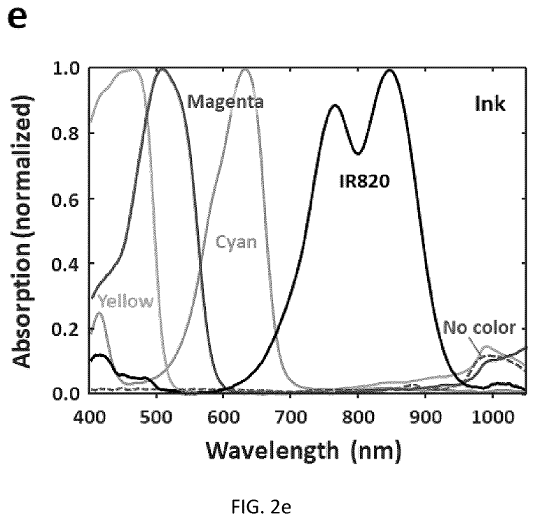

For bioinkjet printing, we used biological safe coloring and NIR absorbing dyes (shown in FIG. 2a, which provides chemical structures of various ink species of interest). Specifically, the main ingredients of color inks include FDA-approved food colorings: FD&C Blue No. 1 (brilliant blue FCF) for cyan, FD&C Red No. 3 (erythrosine), FD&C Red No. 40 (Allura Red AC) for magenta, and FD&C Yellow No. 5 (tartrazine) for yellow. IR820 is the main ingredient for the NIR absorbing invisible ink; IR820 (1 μM) dissolved in deionized water is transparent due to its low absorption in the visible range (FIGS. 2b and 2c which include a photograph (FIG. 2b) and normalized absorption spectra (FIG. 2c) of food colorings and IR820 dye dissolved in deionized water). Here, Blue No. 1 (B#1, brilliant blue FCF), Yellow No. 5 (Y#5, tartrazine), Red No. 3 (R#3, erythrosine), Red No. 40 (R#40, Allura red AC): 10 μM and IR820: 1 μM). The viscosity of all inks is maintained in a range of 3-5 mPa·s (cP) for ensuring that it is comparable to water-based inkjet inks. Colorimetric analyses show that cyan, magenta, and yellow inks have colors corresponding to the absorption spectra of the food colorings (FIGS. 2d and 2e, which include photographs (FIG. 2d) and normalized absorption spectra (FIG. 2e) of color inks (1:10000 diluted) and IR820 inks). Here, color inks: Cyan (Blue No. 1, 50 mM), magenta (Red No. 3, 30 mM and Red No. 40, 100 mM), and yellow (Yellow No. 5, 100 mM)). The ink without any dyes (i.e., no color) is also shown. In FIG. 2d, lower images are captured with an NIR-enabled camera under NIR light illumination (λ=850 nm). In FIG. 2e, the concentration of IR820 ink for the optical absorption measurement is 10 μM. All color inks are clear upon NIR light illumination at a center wavelength of 850 nm. In contrast, the IR820 ink at 1 mM is dark green, while highly concentrated IR820 inks appear black under white light illumination because of strong absorption in the visible wavelength region of 400-650 nm. As expected, all IR820 inks are black under 850-nm NIR light illumination because of the high absorption peak at 850 nm. Therefore, after printing, the invisibility of the IR820 ink is achieved by determining the optimal concentration. Finally, the prepared ink solutions are injected into refillable ink cartridges of a commercially available inkjet printer.

We implemented a QR code as a taggant because the solid dosage formats of medicines and other ingestible products cannot be fed directly into a commercially available inkjet printer to print a QR code onto the surface of medicines. However, with other printing technologies, on-dose printing is possible and thus the QR code can be printed directly onto the ingestible product. It should be noted these two embodiments (i.e., printing onto a substrate and adhering the substrate to the ingestible product vs. printing the QR code directly onto the ingestible product) are within the ambit of the present disclosure. For the first embodiment, a silk fibroin print sheet can easily feed into an inkjet printer. From an edible perspective, the silk fibroin is not only recognized as safe by the FDA as a food additive, but it is also biocompatible, digestible, noncytotoxic, nonantigenic, and noninflammatory. From an optical perspective, silk fibroin exhibits negligible optical absorption in the visible and NIR wavelength range of 400-1000 nm. From a manufacturing perspective, the easy processability of the silk fibroin offers a scalable production option; however, printing fine patterns using water-based inkjet inks on a silk fibroin print sheet is not straightforward because the printing ability is affected by the surface properties of the print sheets. Unlike paper, silk fibroin sheets do not absorb inkjet droplets because of their water-repellent surfaces. The inkjet droplets often spread because of wetting, which results in droplet aggregation.

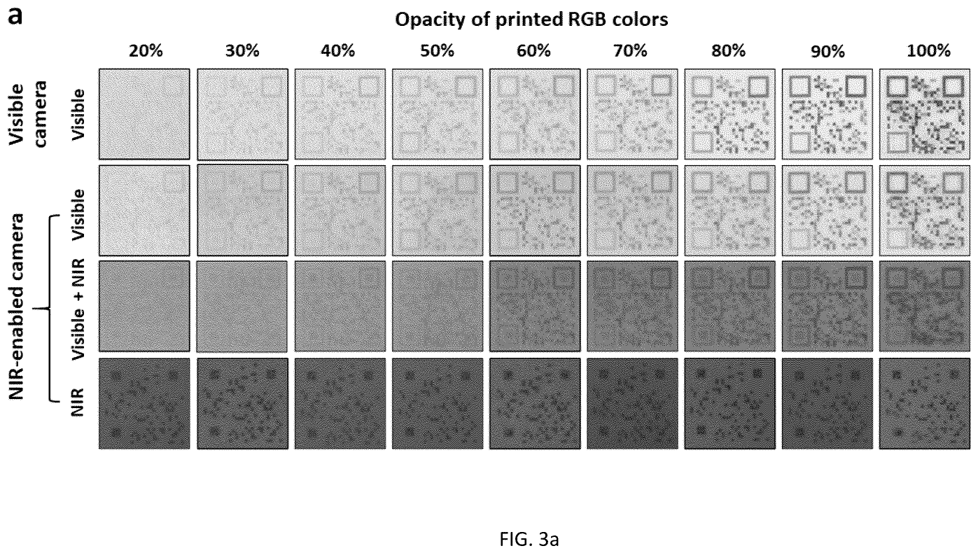

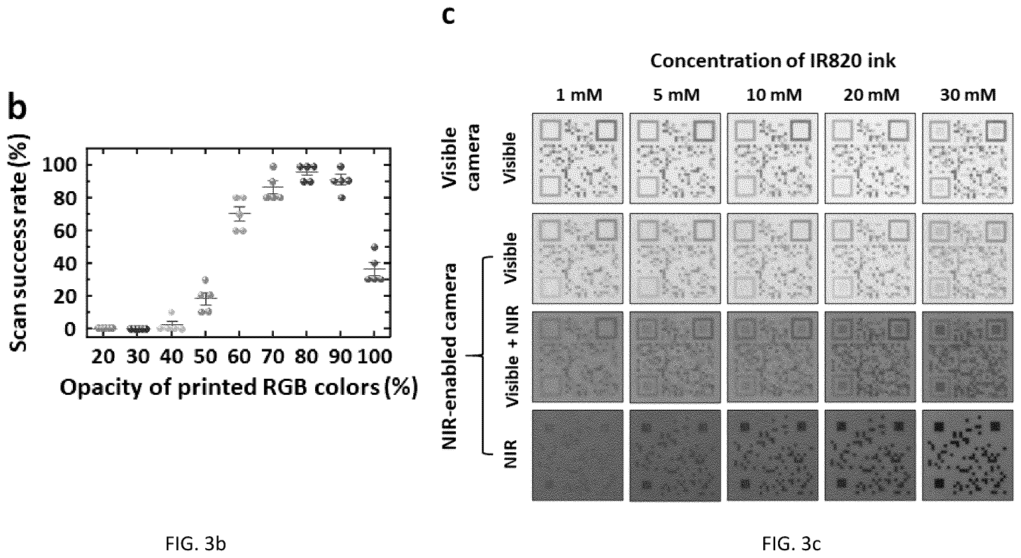

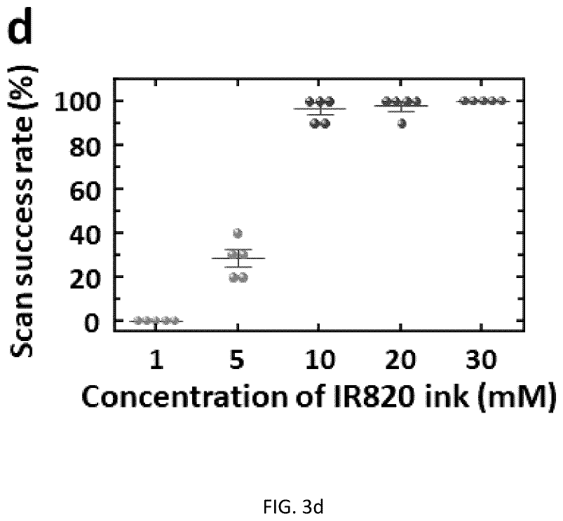

The density of the droplet-jetted inks on silk fibroin print sheets in bioinkjet printing can be controlled by varying the opacity level in digital printing. QR codes with color and NIR invisible patterns are printed on silk fibroin print sheets at different opacity levels of red (R), green (G), and blue (B) colors in a range of 20-100% (see FIG. 3a, which includes photographs of QR codes printed on silk fibroin print sheets with different opacity levels of the RGB colors in a range of 20-100%). The concentration of IR820 ink is kept at 10 mM. Photographs of the QR codes are captured using two smartphones either equipped with a standard visible camera or an NIR-imaging-enabled camera under visible (white) light or/and NIR (λ=850 nm) light illumination. In QR codes, the RGB color pattern is captured by both the visible camera and NIR-enabled camera under visible light illumination, while the invisible NIR pattern printed with the IR820 ink is revealed only under NIR light illumination (λ=850 nm).

We determined the optimal opacity of RGB color printing to be 80% with a scan success rate of 96±4.9% (mean±standard deviation). At lower opacity levels, the absorption spectra intensity of RGB colors is too weak, although the droplets of the color inks are individually well distributed on the silk fibroin print sheet. In this case (<50%), the average scan success rate is low below 18% (FIG. 3b, which is a graph of success rate in % vs. opacity of printed RGB colors in %). At high opacity levels above 90%, the readability of color QR codes decreases because the high density of droplets forms significant aggregations of droplets, and the optimal opacity level for RGB color printing limits the minimum printable size. The minimum size of the reported QR codes can be determined based on a tradeoff between inkjet printing and camera image resolutions, although smartphone cameras have high-performance (optical, digital, or hybrid) zoom options. When QR codes are printed in different sizes and imaged under visible and NIR light illumination, QR codes with sizes larger than 7×7 mm² show scan success rates greater than 90% without using the zoom option of the NIR-enabled camera.

For the IR820 ink, we consider the opacity level of IR820 printing and the concentration of the IR820 ink. An opacity level of 100% is selected to ensure a sufficient printing resolution. Unfortunately, unlike the available control of opacity levels in color printing, the variable opacity level of the black color in digital printing is not implemented on the print sheets using only IR820 ink. At an opacity level of 100%, only the IR820 ink is present on the silk fibroin print sheet, whereas color droplets are printed at opacity levels below 90%, which reveal the absorption in the visible wavelength range. Highly concentrated IR820 ink has a dark greenish color (seen in FIG. 2d), and therefore, its concentration must be optimized to minimize visibility under visible light and maximize visibility under NIR illumination. The optimal concentration of IR820 is determined to be 10 mM for ensuring that the NIR QR code pattern is not visible to the naked eye (see FIGS. 3c and 3d, which are photographs (FIG. 3c) and scan success rate (FIG. 3d) of QR codes printed on silk fibroin print sheets with different concentrations of IR820 inks in a range of 1-30 mM). Scanning tests of five different QR codes are performed, and each QR code is read 10 times repeatedly. The size of the QR code taggants is 9×9 mm². Under NIR light illumination, the print pattern becomes clearer with an increase in the concentration of IR820ink, and this results in the enhanced readability of QR codes with scan success rates of 96-100% above 10 mM. Below 10 mM, the pattern printed in the QR codes using IR820 ink is invisible to the naked eye under visible light illumination; however, it shows greenish colors at 20 and 30 mM. The QR code printed with 30 mM IR820 ink could be read using a QR code scanner under visible light without NIR light.

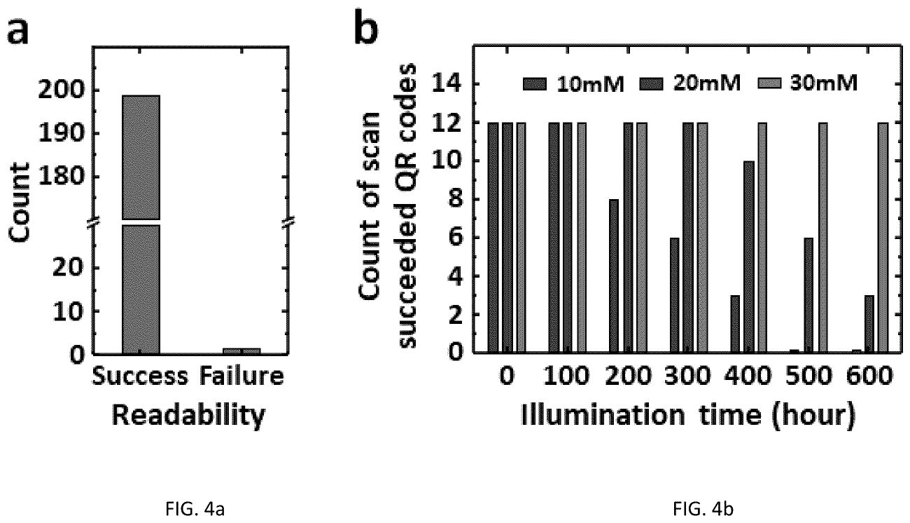

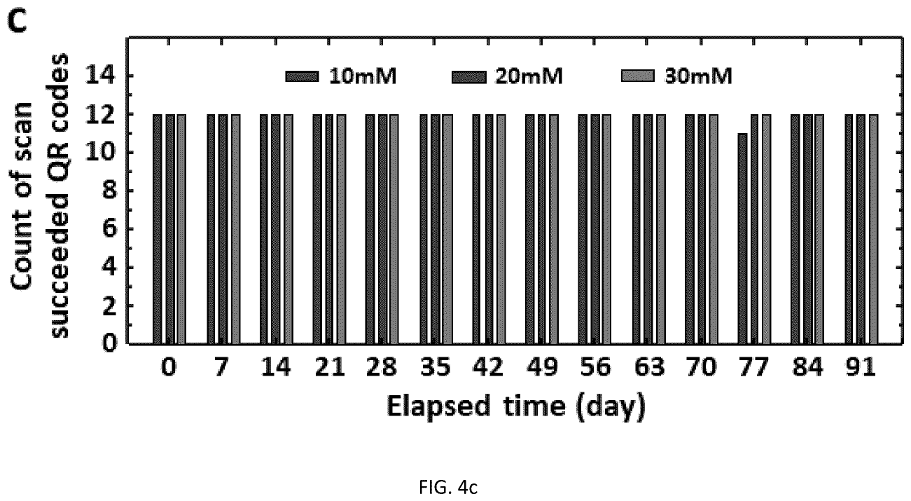

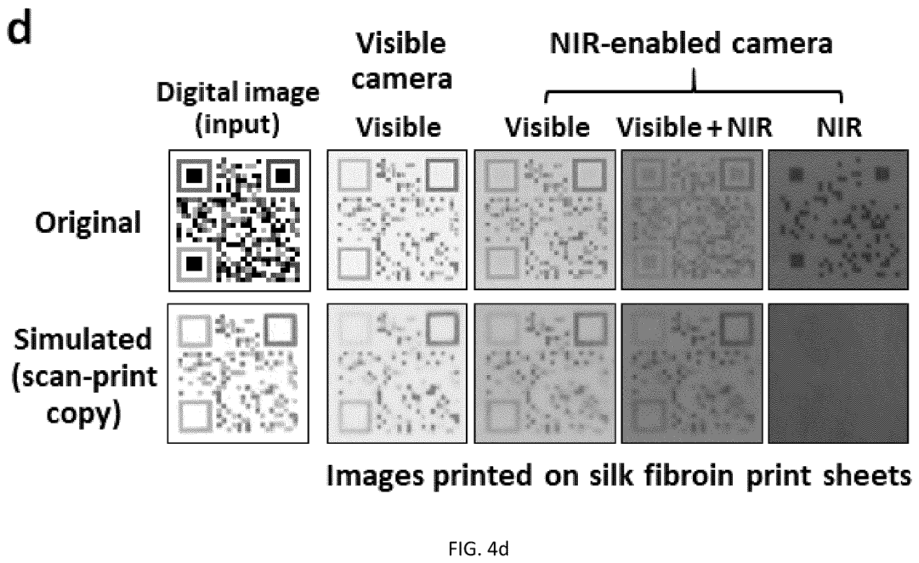

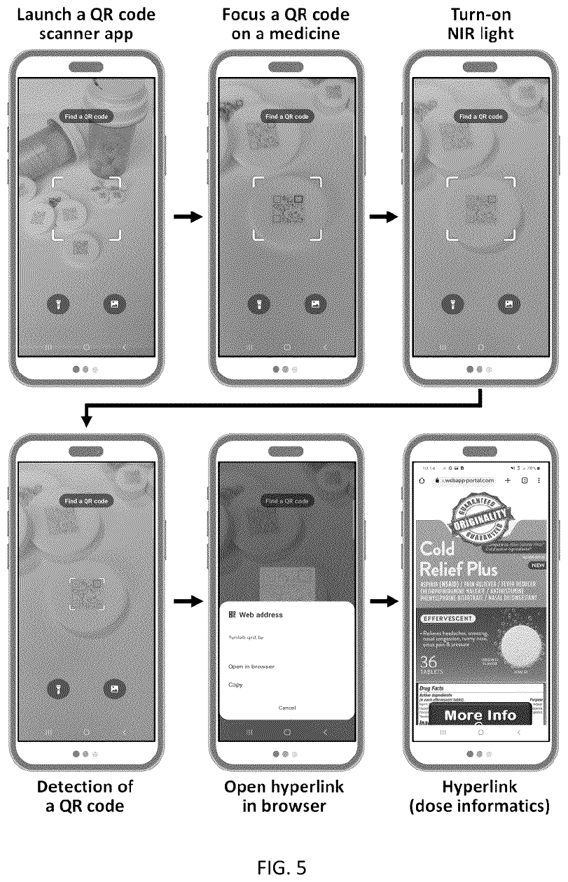

We evaluated the printing reproducibility, photostability, and long-term stability of the QR code taggants. First, the print reproducibility, defined as the ability to produce a large number of identical QR codes on silk fibroin print sheets, is tested by producing 200 different taggants with the same digital QR code (see FIG. 4a, which is a graph of printing reproducibility test of 200 different taggants with the same QR code pattern using bioinkjet printing). A total of 199 QR codes succeeded when read, which results in a printing reproducibility of 99.5%. The reading failure of only one QR code taggant is attributed to the distortion of the printed QR code patterns caused by the uneven surface condition of the print sheet. The photostability of the QR code taggants is tested, and the taggants are illuminated with white light (D65 LEDs) for 600 h at an intensity of 500 lx, which is the recommended office workspace light intensity (FIG. 4b which is a graph showing scan success rates of 12 different QR code taggants printed at different concentrations of IR820 ink for photostability tests). Clearly, the QR code scan success rate decreases with longer durations of light exposure. At a lower concentration of 10 mM, the readability is maintained for up to 180 h, because of the photodegradation of IR820, whereas the three RGB colors maintain their vibrancy for 600 h without undergoing bleaching. The use of commonly available pharmaceutical packaging designed for light protection (e.g., dark or opaque packaging covers) substantially prolongs shelf life. In addition, the edible QR code taggants show good long-term reliability for 91 days, even without the use of protective packaging (see FIG. 4c, which is the same as FIG. 4b but for long-term stability tests).

To ensure the QR code according to the present disclosure is sufficiently robust against scan and copy attacks, where an illicit counterfeiter possesses unrestricted access to the technologies and materials employed to produce the proposed QR code taggants, including IR820 and coloring dyes, identical inkjet printers and cartridges, and silk fibroin print sheets (see FIG. 4d, which provides photographs of original and simulated (scan-print copy) QR code taggants). The scan-printed QR code is not readable by a QR code scanner because of the invisibility of IR820 ink areas in the QR code. The concentration of IR820 ink is 10 mM and the opacity of printed RGB colors is 80%. The QR code taggant size is 10×10 mm². We assume that a counterfeiter uses a high-resolution digital scanner (600 dots per inch (DPI)) to acquire a digital (input) image of a QR code taggant. Subsequently, the counterfeiter reprints the scanned digital QR code image onto a silk fibroin print sheet. The colors and patterns captured by both the standard and NIR-enabled cameras appear similar when comparing the original and copied QR codes under visible illumination. However, under NIR light illumination, the invisible pattern in the scan-printed QR code is not detected because the IR820 ink pattern is not scanned or printed at all, which results in QR code reading failure. Bioinkjet printing technology using invisible IR820 ink can be applied to print completely hidden QR codes on the surface of silk fibroin print sheets included with various colors, and this can potentially be used for other cryptography and security applications.

Next, on-dose authentication is demonstrated using a camouflaged safe QR code taggant affixed to an oral dosage tablet (see FIG. 5, which is a collection of photographs of a smartphone utilized to simulate authentication process of a QR code taggant affixed to an oral-dosage tablet-type medicine). The onboard QR code scanning application in a smartphone includes the following steps: Launch the QR code scanner app, focus the QR code taggant on a medicine, and turn on the portable NIR LED flashlight. The mobile app detects the QR code and further opens the encoded hyperlink to a webpage for confirming the verification and authentication and for providing the medicine information. The QR code taggant size is 10×10 mm². A smartphone equipped with a portable NIR LED flashlight and an onboard QR code scanner is used in the simulated setting. The QR code taggant is attached to a solid-type medicine (Alka Seltzer) using glucose syrup glue (FONDX America Corp.). After launching the QR code scanner app, the end user (e.g., patient or pharmacist) views and focuses the QR code affixed to the solid medicine through the smartphone screen, and then, the user turns on the NIR LED flashlight. The scanner application automatically recognizes the color and invisible QR code patterns, immediately opens the embedded hyperlink to confirm authentication, and provides important dose information. Notably, the camouflaged safe QR codes can only be scanned under both visible and NIR light illumination, which can potentially be used to overcome the limited security of conventional QR code technologies.

With the advent of the taggant printed directly onto an ingestible product or on a substrate that is then affixed to the ingestible product, according to the present disclosure, additional security measures in the form of two-factor authentication can be implemented. For example, once the user scans the QR code on the ingestible product, they can access the manufacturer's website. The manufacturer can then use the user's cell phone number from their database to text a security code, initiating contact with the user. The manufacturer would then require entry of the security code before any other communication can resume. Additionally, with the advent of the presently disclosed taggant, the manufacturer can establish continuous monitoring of the usage of the product. For example, each time a patient is about to ingest a pharmaceutical product, scanning the QR code and establishing communication with the manufacturer or the doctor's website allows the manufacturer or the prescribing doctor to monitor proper compliance with the initial order of the pharmaceutical product. Such monitoring opens the door for feedback from the manufacturer or the prescribing doctor in case of lack of compliance.

The ink preparation is next discussed. Food-safe polysorbate 80, food-grade propylene glycol, and ethyl alcohol (200 proof) were formulated to create inkjet inks using IR820 and FDA-approved coloring dyes. The IR820 dye is dissolved in deionized water at 37° C. for 30 min to prepare the IR820 ink, resulting in concentrations of 1, 5, 10, 20, and 30 mM. The composition of the IR820 ink consists of 85.8% IR820/water, 9% ethyl alcohol, 5% propylene glycol, and 0.2% polysorbate 80. Brilliant blue FCF (Blue No. 1, 50 mM), tartrazine (Yellow No. 5, 100 mM), erythrosine B (Red No. 3, 30 mM), and Allura red AC (Red No. 40, 100 mM) color dye solutions were used as the color inks. Food-safe polysorbate 80, food-grade propylene glycol, and ethyl alcohol were added. Polysorbate 80 acts as a dispersing agent and propylene glycol serves as a carrier to maintain the desired viscosity and prevent the inks from drying. The color dye-dissolved water of 90% was formulated along with 5% ethyl alcohol, 4.9% propylene glycol, and 0.1% polysorbate 80 for color inks: Brilliant blue FCF for cyan, tartrazine for yellow, and a mixture of erythrosine B and Allura red AC (1:3 ratio) for magenta. The prepared ink solutions were injected into refillable ink cartridges, which were subsequently installed in a commercial inkjet printer.

Those having ordinary skill in the art will recognize that numerous modifications can be made to the specific implementations described above. The implementations should not be limited to the particular limitations described. Other implementations may be possible.

1. A method of providing an edible printer dye for use as a predetermined pattern including a stealth quick response (QR) code taggant for an ingestible food or pharmaceutical product, comprising:
dissolving one or more edible infrared (IR) or near IR (NIR) light absorbing dyes in a carrier solution based on a predetermined concentration of the one or more IR or NIR light absorbing dyes, thereby generating an IR or NIR light absorbing dye solution;
adding a mixture of dispersing agents to the dye solution, thereby generating an edible IR or NIR light absorbing dyes;
loading the edible IR or NIR light absorbing dyes in any one of a printer cannisters; and
loading other visible edible dyes in corresponding other printer cannisters.

2. The method of claim 1, wherein the one or more edible IR or NIR light absorbing dyes includes indocyanine green (ICG) having a chemical structure:

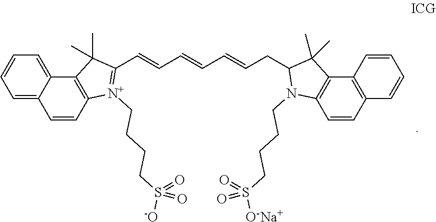

3. The method of claim 1, wherein the one or more edible IR or NIR light absorbing dyes includes IR820 having a chemical structure:

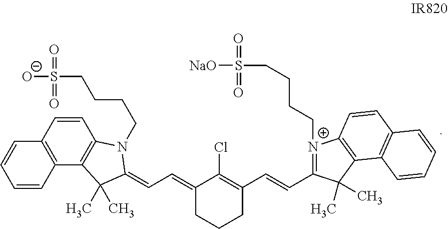

4. The method of claim 1, wherein other visible edible dyes includes an edible blue dye is brilliant blue FCF having a chemical structure:

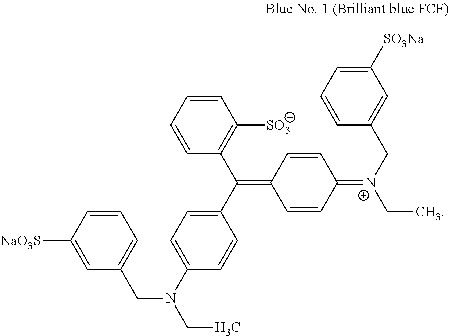

5. The method of claim 1, wherein other visible edible dyes includes an edible yellow dye is Tartrazine having a chemical structure:

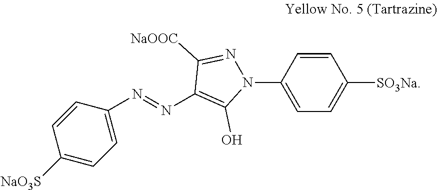

6. The method of claim 1, wherein other visible edible dyes a first edible red dye is Erythrosine having a chemical structure:

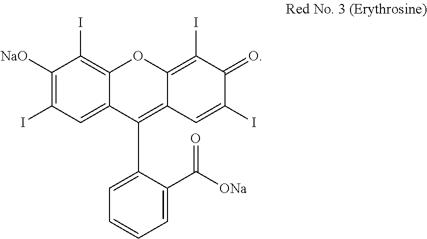

7. The method of claim 6, further comprising:
loading a second edible red dye into a corresponding red cannister.

8. The method of claim 7, wherein the second edible red dye is Allura Red AC having a chemical structure:

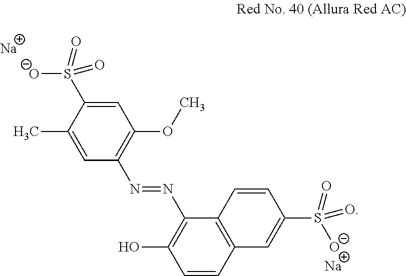 [SMILES: [Na+] ; COc1cc(S(=O)(=O)[O-])c(C)cc1/N=N/c1c(O)ccc2cc(S(=O)(=O)[O-])ccc12 ; [Na+]]

9. A method of generating an edible and stealth predetermined pattern on an ingestible product, comprising:
printing a predetermined pattern including a quick response (QR) code onto an edible substrate; and
adhering the substrate to an ingestible product, the predetermined pattern having a pattern derived from:
one or more visible light absorbing dyes, and
one or more edible infrared (IR) or near IR (NIR) light absorbing dyes, wherein the one or more edible IR or NIR light absorbing dyes are based on dissolving one or more edible IR or NIR light absorbing dyes in a carrier solution based on a predetermined concentration of the one or more edible IR or NIR light absorbing dyes, thereby generating an edible IR or NIR light absorbing dye solution;
adding a mixture of dispersing agents to the dye solution, thereby generating one or more edible IR or NIR light absorbing dyes.

10. The method of claim 9, wherein the substrate is a protein-based print sheet.

11. The method of claim 10, wherein the protein-based print sheet is a silk fibroin print sheet.

12. The method of claim 10, wherein the protein-based print sheet is a gelatin print sheet.

13. The method of claim 10, wherein the protein-based print sheet is an edible print paper or edible image sheet.

14. The method of claim 9, wherein the QR code is printed directly onto the ingestible product, the ingestible product including a pharmaceutical product.

15. The method of claim 9, wherein the one or more edible IR or NIR light absorbing dyes includes indocyanine green (ICG) having a chemical structure:

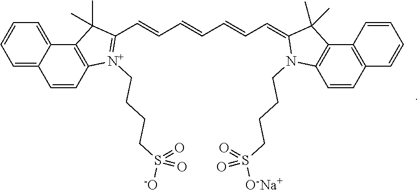

16. The method of claim 9, wherein the one or more edible IR or NIR light absorbing dyes includes IR820 having a chemical structure:

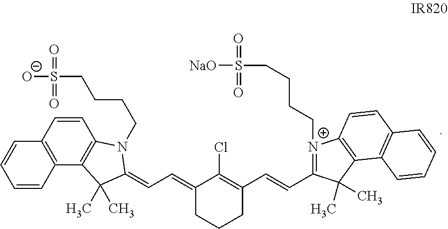

17. The method of claim 9, wherein the one or more edible visible light absorbing dyes includes an edible blue dye, an edible yellow dye, and a first edible red dye.

18. The method of claim 15, wherein the edible blue dye is brilliant blue FCF having a chemical structure:

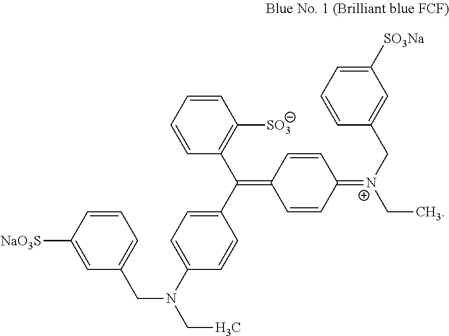

19. The method of claim 15, wherein the edible yellow dye is Tartrazine having a chemical structure:

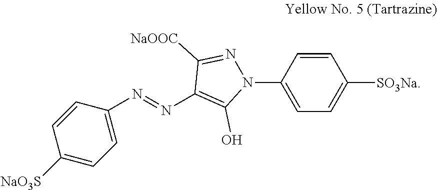

20. The method of claim 15, wherein the first edible red dye is Erythrosine having a chemical structure: [chemical structure]
